(19) United States
(12) Patent Application Publication
(10) Pub. No.: US 2011/0165620 A1
(43) Pub. Date: Jul. 7, 2011

(54) METHOD FOR THE PRODUCTION OF PROTEINS OR PROTEIN FRAGMENTS

(76) Inventors: Roland Romijn, Tiel (NL); Weiger Hemrika, Amsterdam (NL)

(21) Appl. No.: 12/995,557

(22) PCT Filed: May 28, 2008
(86) PCT No.: PCT/NL08/00137

§ 371 Date: Mar. 4, 2011

(51) Int. Cl. C12P 21/02; C40B 30/06; C12N 15/63; C12N 5/071; C12N 15/85

(52) U.S. Cl. 435/69.1; 506/10; 435/320.1; 435/369; 435/455

(57) ABSTRACT

The present invention relates to a method for selecting a suitable expression construct from a plurality of expression constructs for optimizing the production of a protein or a fragment thereof in a host cell, a method for the production of proteins or fragment thereof using the selected expression vector, to novel human embryonic kidney cells that are deficient in N-acetyl-glucosaminyltransferase I and stably transfected with EBNA (HEK 293E GnTI′cells) that are well suitable for use in the said method, in particular for the production of proteins or protein fragments that are suitable for X-ray studies. The invention also relates to a method to produce HEK 293E GnTI′ cells and a method to confer to HEK293E GnTI′ cells, the capacity to grow in suspension and to a method to confer to HEK293E GnTI′ cells, the capacity to grow in serum free medium. The invention also relates to a kit comprising different vectors suitable for use of the above method for the production of proteins or protein fragments.

The present invention relates to a method for selecting a suitable expression construct from a plurality of expression constructs for optimizing the production of a protein or a fragment thereof in a host cell, to a method for the production of a protein or a fragment thereof using the selected expression vector, to novel Human embryonic kidney cells that are deficient in N-acetylglucosaminyltransferase I and stably transfected with EBNA1 (HEK 293E GnTI⁻ cells) that are well suitable for use in the said method, in particular for the production of proteins or protein fragments that are suitable for X-ray studies. The invention also relates to a method to produce HEK 293E GnTI⁻ cells and a method to confer to HEK293E GnTI⁻ cells, the capacity to grow in suspension and to a method to confer to HEK293E GnTI⁻ cells, the capacity to grow in serum free medium. The invention also relates to a kit comprising different vectors suitable for use of the said method for the production of proteins or protein fragments.

Proteins, including recombinant proteins and fragments thereof, are useful for e.g. scientific, therapeutic, nutraceutical and industrial applications. In the art, there is a continuous desire to improve the production of proteins. Several heterologous overexpression systems have been developed to produce these proteins and fragments thereof, and each has its advantages and drawbacks. For example, proteins and protein fragments can be produced by bacteria such as Escherichia coli. To this end, the gene encoding the protein (or fragment thereof) of interest is introduced into E. coli and expressed to produce the envisaged protein. This system is fast, low-tech, cheap and easily scalable. However, the major drawback using E. coli expression systems is the lack of post-translational modifications like disulfide bridge formation, glycosylation, sulfation and phosphorylation. Prokaryotic overexpression systems are usually the system of choice for the production of single domains or small single domain proteins that do not require post-translational modifications. Popular yeast overexpression systems are Pichia pastoris and Saccharomyces cerevisiae. Yeast expression systems have same advantages as prokaryotic expression systems and are capable of some post-translational modifications. However, yeast overexpression systems occasionally fail to produce complex and multidomain mammalian proteins. Insect cell overexpression systems (for example HighFive cells, SF9 cells) are capable of post-translational modifications, but glycosylation chains are different compared to glycosylation chains produced by mammalian cells. Other disadvantages are high running costs, time consumption and the requirement of relative expensive laboratory equipment. Most complex recombinant overexpression systems are mammalian expression systems. In EP1390511B1, of which Durocher is the first inventor, an expression vector for improved production of recombinant proteins by transient expression in human embryonic kidney cells is described. To arrive at the higher expression, the cells stably express EBNA1 protein and the vector wherein the gene of interest is present comprises the oriP sequence of the Epstein-Barr virus. The gene is under the control of the CMV5 promoter.

The problem with existing overexpression systems such as that of EP1390511B1 is that effective production of the envisaged protein by the used expression system is not predictable; it may very well be that a protein is not well expressed in a particular expression system, but well expressed in another. In fact, EP1390511B1 only shows improved expression for two proteins, namely human placental secreted alkaline phosphatise (SEAP) and green fluorescent protein (GFP).

Successful protein production however, depends on the combination of many variables, such as the copy number of the introduced gene, the choice and presence of elements affecting the transcription of the gene, such as e.g. promoters and enhancers. Also sequence elements affecting proper excretion, such as signal sequences can be decisive in the success of proper expression of the envisaged gene to produce the protein of interest (or fragment thereof). In particular when only a fragment of a protein is to be produced, such as a particular protein domain, proper folding of the said domain may be important to produce the said protein fragment in proper fashion. Elements affecting proper folding should be present on the encoding DNA. Such elements may e.g. be other portions of the same protein of the envisaged protein fragment, or may originate from other proteins. Also, it may be desired to produce a recombinant protein that comprises additional sequences, for example to enable convenient detection or purification of the protein (fragment). The presence of such a detection/purification tag may very well affect the expression of the gene and the production of the protein (fragment).

Therefore, the production of an envisaged protein by expressing the encoding gene in a suitable expression system is laborious and not straight forward.

Even closely related domains within the same protein may show great variety in expression and production, even when expression takes place in an optimised expression system as that of EP1390511B1. Morlot et al., Acta Cryst. (2007), D63, pp. 961-968, describe the production of four Slit2 LRR domains in a mammalian expression system. The domains were cloned into vectors, similar to that of EP1390511B1 but comprising additional sequences that might have an effect on the expression of the domains. The domains were expressed, either combined with a cystatin signal peptide, or an artificial signal peptide together with a C-terminal hexahistidine tag, or a full length human growth hormone in combination with a TEV cleavable hexahistidine tag. It was found that in the chosen settings, expression of three of the four domains was less critical. However, one of the domains (Slit2 D1) appeared to be only produced when combined with the full length human growth hormone sequence.

So in the art, a major problem exists when proteins or fragments thereof are to be produced by expressing the encoding gene in producing host cells. Even closely related protein domains are produced in a very variable manner when using the same cloning conditions.

In the art, solutions are proposed by improving additional variables. Durocher, inventor of EP1390511B1, proposes to improve the expression further, i.e. in addition to the above discussed improved expression vector, by improving the culture medium and the transfection process (Nucl. Ac. Res. (2002) Vol. 30, No. 2. e9).

The present invention contributes to the solution of the above problem by realizing that the production of proteins seem to be affected significantly by the presence, absence, and location of additional sequences in the expression vector. The invention avoids the problem of the laborious preparation of different expression vectors for each DNA to be expressed, and provides an elegant solution to select the most suitable vector for the optimal production of the envisaged protein or fragment thereof.

This invention relates in a first aspect to a method for selecting a suitable expression construct from a plurality of expression constructs for optimizing the production of a protein or a fragment thereof in a host cell, the fragment not being a Slit2 LRR domain, comprising the following steps:
a) providing a first and a second DNA construct, each comprising
   a common vector sequence,
   a common cloning site,
   a DNA encoding the protein or fragment thereof,
   the constructs being different in sequence, location or presence of a DNA sequence element affecting the production of the protein or fragment thereof by the envisaged host cell,
b) providing host cells, and transfecting a first portion of the host cells with the first construct obtained in step a), resulting in first transfected host cells, and transfecting a second portion of the host cells with the second construct obtained in step a), resulting in second transfected host cells,
c) culturing the transfected host cells of step b) under conditions allowing the production of the protein or fragment thereof by the transfected host cells,
d) determining the amount and/or quality of the protein or fragment thereof, produced by the first and second transfected host cells,
e) selecting the host cells producing the highest amount or quality of the protein or protein fragment as determined in step d),
f) selecting the DNA construct used for transfection of the host cells as selected in step e) as the suitable expression construct.

In the first step a), a first and second DNA construct are provided, that each comprises a common vector sequence with a common cloning site. This means that the different DNA constructs are based on the same vector.

A vector can be a plasmid, phagemid, phage, cosmid, a yeast artificial chromosome or a linear DNA vector. In a preferred embodiment of the invention, the vector, is a plasmid.

The constructs have a common cloning site, in particular a restriction enzyme recognition site. This cloning site is intended to be used for cloning the DNA encoding the protein or fragment thereof to produce the envisaged constructs. The common cloning site can be present in the common vector sequence.

Preferably, the common cloning site comprises multiple different restriction enzyme recognition sites. The advantage thereof is that each construct comprises multiple different restriction enzyme recognition sites, providing more choice for cloning the envisaged DNA fragment into the cloning site. E.g. by using different restriction sites for the 5′ and 3′ end of the DNA encoding the protein or fragment thereof, the orientation of the said DNA in the construct can be conveniently chosen.

The constructs comprise the DNA encoding the protein or fragment thereof that is to be produced. This DNA is also identical in the different constructs, and is cloned into the common cloning site of the constructs. As the said common cloning site is also identical among the constructs, the DNA encoding the protein or fragment thereof can be conveniently cloned to produce the different constructs.

Importantly, the constructs differ from one another in the sequence, location or presence of a DNA sequence element affecting the production of the protein or fragment thereof by the envisaged host cell. As explained above, such sequence element can be a promoter or enhancer (i.e. a non encoding structural DNA element), or can encode a signal sequence or another additional sequence, i.e. involved in excretion or proper folding of the protein or fragment thereof. Thus, the first DNA construct may comprise a sequence element encoding a signal sequence, whereas the second DNA construct may comprise another sequence element encoding another signal sequence, or the second DNA construct would not have such an element. The first DNA construct may also comprise an enhancer at a particular location, whereas the second DNA construct would comprise the same enhancer at another location in the construct, or would not have the said enhancer, or would have another enhancer. Or the first DNA construct can have a detection/purification tag, whereas the second DNA does not, or has another tag, or has the same tag at a different location (e.g. 3′ of the DNA encoding the protein or fragment thereof in the first DNA construct, and 5′ of the DNA encoding the protein or fragment thereof in the second DNA construct), or combination of these differences. It is also possible that the one or more DNA constructs comprise more than one such DNA sequence element.

By this, the first and second construct differ from one another in one or more DNA sequence elements, but share the DNA encoding the protein or fragment thereof, the vector sequence and the common cloning site. In preparing the different constructs, the DNA encoding the protein or fragment thereof is cloned into the common cloning site and does therefore not need different treatment for the different constructs. This facilitates the production of the different constructs significantly. For example, when the constructs have a BamH1 site as common cloning site, the DNA encoding the protein or fragment thereof should contain ends, compatible to BamH1, to be cloned into all the different constructs, without the need for additional treatment for one or more different constructs. Thus, the DNA encoding the protein or protein fragment can be treated only once to produce the correct ends, which enables universal ligation into the universal cloning site.

Herein, the term ‘protein’ is meant to include any protein or protein fragment that is encoded by the DNA coding for the envisaged protein or protein fragment. The protein can be an endogenous protein for the host cells, or can be exogenous, or recombinant. It is also referred herein as ‘envisaged protein’.

The DNA coding for the protein or protein fragment may also be referred to herein as ‘insert’ or ‘encoding DNA’. For cloning reasons, the said DNA coding for the protein or protein fragment is provided as an insert ready to be cloned into the common cloning site to produce the different constructs. The insert may also contain additional sequences encoding adjacent amino acids of the envisaged protein fragment to be produced. Such additional sequences may have a positive effect on proper folding of the envisaged protein fragment.

In the next step b), host cells are provided and transfected with the DNA constructs obtained as described above. The different DNA constructs are transfected into different portions of the host cells, to allow the different transfectants to be grown in separate containers, such as multi-well petri dishes, flasks etc. The host cells can be any host cells which can be used to express the envisaged DNA encoding the protein or fragment of interest resulting in production thereof. Transfection methods are well known in the art and a skilled person in the art will be able to select the correct transfection method that suits the host cells and the vector best. In an attractive embodiment, the transfection is performed using polyethyleneimine (PEI) as a transfection agent. The inventors have shown that high transfection efficiencies can be obtained by using PEI.

In the following step c), the transfected host cells of the previous step are cultured under suitable conditions to allow the cells to express the encoding DNA and produce the envisaged protein or protein fragment. Preferably, the conditions are identical for the different (i.e. the first and second) transfected host cells.

In the subsequent step d), the amount and/or quality of the proteins or protein fragments, produced by the different transfected host cells is determined. It may be important to not only consider the amount of the produced protein or fragment thereof, but also to consider other quality aspects, such as proper folding, ability to be purified, apparent mass of the molecule etc. Also the ability to be excreted is important in this respect.

The expression level can be determined using well described methods from the art. Also, the purity or other quality aspects of the protein can be determined. For example it may be determined whether the protein contains a specific post translational modification. Methods to analyse protein levels are well known in the art.

In the next step e), the host cells that produce the highest amount and/or quality of the envisaged protein or fragment thereof as determined in the previous step are selected. Therein, a comparison of the production level or quality as determined in the previous step can be made, and the best producer is selected.

In step e), the transfected host cells are selected. As these cells were transfected with a particular DNA construct, It is easy to determine and select in a last step f) the construct used for the transfection of the best producing cells. This construct is therewith the most suitable expression construct for the production of the envisaged protein or fragment thereof.

The method allows the preparation of a collection of DNA pre-constructs, i.e. constructs wherein only the DNA encoding the protein or fragment thereof is still to be cloned to obtain the different corresponding DNA constructs, ready for testing according to the invention. This collection can be used to prepare a custom designed DNA construct for any DNA, encoding a protein (fragment) of interest. Such a fragment can e.g. be provided by a customer. The method according to the invention is performed with two or more of the pre-constructs from the collection, and the corresponding DNA construct, leading to the best producing transfected host cells is selected and proposed to the customer.

The skilled person is aware of suitable techniques necessary to perform the methods according to the present invention, e.g. for preparing DNA encoding a protein or fragment thereof of interest, cloning and of any other techniques used in the field of biotechnology such as screening methods, transfection and growing of cells. In addition, reference is made to standard literature, such as Sambrook and Russel, Molecular cloning, a Laboratory Manual, Cold Spring Harbor Laboratory Press, 2001, ISBN 0879695773, and Primrose and Twyman, Principles of Gene Manipulation and Genomics, Blackwell Science, 2006, 7^{th} Edition, ISBN 1405135441.

The method allows for the simultaneous small scale testing of protein production by the use of multiple different expression vectors as outlined above. The construct that best fulfils the particular needs (e.g. highest expression level, highest purity, presence or location of a purification tag etc.), can than be chosen for large scale protein production. In this way the whole process from vector construction, small scale testing and up to large scale production may be pursued in a short time period, e.g. 3-6 weeks.

In step a) of an attractive embodiment of the method according to the invention, n different expression constructs are provided, in step b) n portions of the host cells are provided, which are transfected with the n expression constructs, resulting in n different transfected host cell portion, and in step d) the amount and/or quality of the protein or fragment thereof, produced by the n different transfected host cell portions is determined.

In this embodiment, more than two different constructs are used. In general, if more vectors are used, a wider range of relevant variables can be tested, increasing the chance that host cells are obtained having optimally improved production level of the protein (fragment) of interest. Thus, an optimally suitable expression construct can be selected by additional transfections of different constructs in parallel.

Preferably, 3 or more different constructs are prepared, resulting in first, second and third, and optionally more different transfected host cells are obtained, among which the best producer, and therewith also the corresponding DNA construct is selected. The integer n is therefore preferably 3 or more, more preferably 4 or more, even more preferably up to 20 inclusive, most preferably between 4 and 10.

Optimized production of the envisaged protein or fragment thereof can be achieved by using the expression construct selected according to the above to be the most suitable. To this end, the invention also relates to a method for the production of a protein or fragment thereof, comprising the following steps:
I. transfecting host cells with a construct, selected according to the above,
II. culturing the transfected host cells under conditions allowing the production of the protein or fragment thereof in the transfected host cells,
III. harvesting the produced protein or fragment thereof from the transfected host cells of step II.

The selected construct is used to transfect a preparative amount of host cells, preferably the same host cells as used for the selection of the most suitable expression construct. The said cells are cultured under suitable preparative conditions, as known to the skilled person, allowing the production of the protein (fragment), where after the produced protein (fragment) is harvested from the host cells, and optionally further purified or isolated. In case the protein (fragment) is excreted into the medium, it can be conveniently purified from the medium. Intracellular produced proteins or fragments can be purified from e.g. lysed cells.

Alternatively, the protein can be produced by further culturing the transfected host cells used to select the most suitable expression construct. To this end the method for the production of a protein or fragment thereof, comprises the following steps:
A. culturing the selected host cells of the above step e) under conditions allowing the production of the protein or protein fragment in the host cells, and
B. harvesting the protein or protein fragment produced by the selected host cells.

This approach avoids an additional transfection step, and allows continuation of growing the best producing cells for production of the protein (fragment).

In a preferred embodiment, the protein or protein fragment is produced by the host cells by transient expression of the DNA encoding the protein or fragment thereof. As outlined above, transient expression has been shown to be a powerful method to produce proteins, in particular mammalian proteins by mammalian cells, more preferably human proteins by human cells. Reference is made to Durocher et al., supra.

Preferably, the DNA sequence element affects the expression level of the protein or protein fragment. Such an element results in enhancing transcription of the encoding DNA and/or the translation of the corresponding mRNA into the protein (fragment). Examples of such elements are promoters, enhancers, and Kozak sequences.

Preferably, such a DNA sequence element comprises a promoter. Promoters are known to be of great effect to the transcription of genes. Hence, the presence of a suitable promoter influences the production level of a protein. It is therefore an advantage to include variety in the promoters driving the transcription of the encoding DNA. According to the method of the invention, the most suitable promoter will contribute to the protein production and therewith to the selection of the best producing cells and expression construct. Any promoter known to be effective in the host cells may be used. Promoters may also be used in combination with enhancers. Different combination can be tested in the method according to the invention.

Preferably, the promoter of a DNA sequence element comprises a CMV or an SRalpha or murine metallothionein promoter. The inventors have found that these promoters enhance the transcription effectively and result in high protein levels in a plurality of human host cells. Most preferably, the promoter is a CMV promoter. An immediate early enhancer can be used to even further enhance the CMV promoter activity.

In a preferred embodiment, the DNA sequence element is located adjacent to the DNA encoding the protein or fragment thereof, and encodes an amino acid sequence element so that, when the protein or fragment thereof is produced by the host cells, the said amino acid sequence element is linked to the protein or fragment thereof. In this embodiment, at least one of the DNA constructs is designed such, that the DNA encoding the envisaged protein (fragment) is situated adjacent to other coding sequences, so that, upon translation, the envisaged protein (fragment) is translated as part of a larger protein (fragment), or fusion protein. According to this embodiment, the DNA sequence element can encode a fusion partner, such as, e.g. a signal peptide, and thus, the effect of the presence of a particular signal sequence on the production of the envisaged protein (fragment) can be evaluated, or the effect of different signal sequences can be tested. However, the effect of the presence and/or location of any protein sequence can thus be evaluated with regard to the production of the envisaged protein. It may very well be that particular amino acid sequences facilitate proper folding or excretion of an adjacent protein (fragment).

In an attractive embodiment, the said amino acid sequence element promotes the secretion of the produced protein or protein fragment. Secretion of the protein (fragment) greatly facilitates the isolation and therefore facilitates the production thereof in host cells, because the product(s) can conveniently be isolated from the medium in which the host cells are cultured.

Preferably, this amino acid sequence element is chosen from the group comprising a signal peptide, a growth hormone or functional analogues thereof, an interleukin or functional analogues thereof, and more preferably comprises a signal peptide. Signal peptides are short (mostly 3-60 amino acids long) peptide chains that direct the post-translational transport of a protein. Some signal peptides are cleaved from the protein by signal peptidase after the proteins are transported. Signal peptides generally drive the secretion of a protein and may therefore have a positive effect on the production of an envisaged protein (fragment). Growth hormones or interleukins that are fused to the envisaged protein (fragment) may serve a similar function and may therefore advantageously be used in the method according to the invention. Reference is made to Morlot, supra, showing that one of the Slit2 LRR domains was only produced to detectable amounts when fused to the growth hormone sequence, whereas for other domains, this was of no influence. An example of enhanced secretion by the use of a protein fused with an interleukin is provided by Michael J. Liguoriet et al. Hybridoma. 2001, 20(3): 189-198. Functional analogues of growth hormones or interleukins are protein sequences that are homologous, in particular more than 80%, preferably more than 90% and most preferably more than 95%, with the (parent) protein sequences of a growth hormone or an interleukin, and have similar effect as the parent growth hormone or interleukin, i.e. with regard to affecting secretion levels of the envisaged protein or protein fragment.

Preferably, the signal peptide is chosen from the group consisting of artificial signal peptides (Barash et al, Biochemical and Biophysical Research Communications, 2002, 294 (4), 835-842), Cystatin S, in particular human (Barash et al, supra), Von Willebrand factor (VWF), in particular human (Verweij, EMBO journal, 1986, 5 (8), 1839-1847), or IgK, in particular from Mus musculus. Cystatin S has the following amino acid sequence: MARPLCTLLLLMATLAGALA, Von Willebrand factor has the following amino acid sequence: MIPARFAGVLLALALILPGTLC or MIPARFAGVLLALALILPGTGS and IgK has the following amino acid sequence: METDTLLLWVLLLWVPGSTGD.

More preferably, the artificial signal peptide has the amino acid sequence MWWRLWWLLLLLLLLWPMVWA (SEQ ID. No. 1) or MRPWTWVLLLLLLICAPSYA (SEQ ID. No. 2).

In another embodiment, the amino acid sequence element enables the identification, isolation or monitoring of the protein or protein fragment. Such elements may facilitate identification of the protein (fragment) e.g. in gels or in cells or it may facilitate isolation from crude material such as culture medium or cell lysate or monitoring of the protein in cells and facilitate the production.

More preferably the amino acid sequence element comprises a detection/purification tag. Such tags are peptide sequences linked to the protein. Often these tags are removable by chemical agents or by enzymatic means, such as proteolysis or protein splicing. Preferred examples of such tags are histidine tags, affinity tags, in particular immuno affinity tags and fluorescent tags. Affinity tags are linked to proteins so that they can be purified from their crude biological source using an affinity technique. Examples of affinity tags include chitin binding protein (CBP), Fc-tag, maltose binding protein (MBP), and glutathione-s-transferase (GST). Fluorescent proteins as for example Green Fluorescent Proteins (GFP) or mutants thereof (comprising colour mutants), can be used to monitor a protein using fluorescent microscopy. The presence of fluorescent proteins can also be useful for the determination of protein levels or selecting positive cells using FACS (Fluorescent-activated cell sorting). Histidine tags are well known from the art and can be used to purify proteins using commercially available purification kits. The poly(His) tag is the most widely-used protein tag and it binds to metal matrices.

The histidine tag preferably comprises a polyhistidine stretch of at least 5 histidines, preferably 6 to 8 histidines. Such histidine stretches have been proven very useful for purification of a protein (fragment), containing such a polyhistidine tag. The tag may also be longer than 8 histidine residues.

In an attractive embodiment, the protein or fragment thereof and the amino acid sequence element, linked thereto constitute a fusion protein. The amino acid sequence element may originate from another protein, such as the human growth hormone, Fc, GFP (or mutants thereof) or interleukin, as discussed above. Linked to the protein (fragment) of interest, the advantageous function can be obtained, such as additional stability to the protein (fragment), or the fusion protein may be excreted whereas the envisaged protein without the fused amino acid sequence may be inadequately excreted (as is e.g. valid for the Slit2 D1 domain (Morlot, supra)).

In view of the relative small size and high effectiveness in excretion of envisaged proteins and fragments thereof, a very attractive amino acid sequence element, to be used in the method of the invention, comprises a growth hormone, preferably human growth hormone, or functional analogue thereof.

In a very attractive embodiment, the amino acid sequence element comprises a protease cleavage site. The envisaged protein can be produced linked to an additional amino acid sequence, or as a fusion protein, which additional sequence, or fused portion can be cleaved off by protease treatment, e.g. after purification of the (fusion) protein. It may therefore be very advantageous to use, in the method according to the invention, at least a DNA construct having an additional nucleic acid sequence encoding an additional amino acid sequence and a protease cleavage site, capable to be cleaved off by a protease.

Preferably, the protease cleavage site is cleavable by a protease, chosen from the group, consisting of TEV, thrombin, precision protease, enterokinase and factor X. These proteases are highly specific thereby reducing the risk of aspecific cleavage of the recombinant protein.

In particular when the production of protein or fragment thereof is limited to a fragment of the said protein, i.e. when only a protein fragment is to be produced, such as a protein domain of interest, it may be important for the said protein fragment to be accompanied by flanking amino acid sequences, that are also part of the original native protein. For example, when a protein domain, having amino acids 20 to 32 of a native protein (of e.g. 55 amino acids) is to be produced, it may be advantageous to link the said domain with amino acids from the same protein, such as the N terminus (e.g. amino acid residues 1-15) or the C terminus thereof (e.g. amino acid residues 40-55). The envisaged protein fragment can be protected this way by proteolytic attack, or be better excreted etc. Thus, the amino acid sequence element therefore preferably corresponds to a portion of the same protein, so that the said protein fragment, when produced by the host cells, is linked to the said portion. According to this embodiment, it is also possible that the amino acid sequence element comprises the same amino acid sequence as the protein fragment itself, resulting in a tandemly arranged double fragment. In accordance with the above, there can be a protease cleavage site between the fragment and the amino acid sequence element.

Advantageously, the amino acid sequence element comprises a portion of the protein that is, in the native protein, adjacent to the fragment of the said protein. In this embodiment, the above protein domain of amino acids 20-32 would e.g. be linked to an amino acid sequence element corresponding to e.g. amino acids 5-19, or 33-40 of the same protein. By this, the importance of adjacent amino acids in the protein of the envisaged protein fragment with regard to production of the said fragment can be assessed. To prepare the corresponding DNA constructs, a longer portion of the encoding sequence can be cloned into one of the vectors, whereas another DNA construct can be produced by cloning only the DNA sequence, encoding the envisaged protein fragment. It is however also possible to provide the adjacent amino acid sequence by incorporation of the corresponding encoding sequence in the vector, and to clone the DNA sequence encoding the protein fragment therein. However, in another attractive embodiment, the amino acid sequence elements present in the constructs to be used in the method according to the invention do not contain amino acid sequences, originating from the same protein as the envisaged protein fragment, in particular not those sequences, being, in the native protein, adjacent to the amino acid sequence of the envisaged fragment.

The DNA sequence element is preferably located in the DNA construct such, that the amino acid sequence element encoded thereby is linked to the N terminal or C terminal of the protein or fragment thereof, when produced by the host cells. In this embodiment, the amino acid sequence element is linked to the N or C terminal of the protein, therewith providing the presence of the original terminus of the protein, when produced by host cell according to the invention.

In a preferred embodiment of the invention the position of the nucleic acid sequence element of at least one of the first DNA construct is located downstream to the cloning site, while the second DNA construct comprises the said nucleic acid sequence element upstream to the cloning site. In particular when the nucleic acid sequence element encodes for an amino acid sequence element as outline above, this results in proteins of which the first has a certain functional element at its C terminus, while the second has the same functional element at its N terminus. Accordingly, in the method according to the invention it can be elegantly tested whether the position of such an amino acid sequence element has an effect on the production of the envisaged protein. This is particularly true when the amino acid sequence elements encode a detection/purification tag. For example, it is known that the position (on N-terminus or C-terminus) of a histidine tag affects the expression level and/or the functionality of the protein. This is illustrated in the examples below.

Preferably, the host cells in the method of the invention are eukaryotic cells. Eukaryotic cells are capable of production of multi-domain proteins and have far more abilities for post translational modification than for example prokaryotic cells.

More preferably, the host cells are human cells. For the production of human proteins, the use of human host cells is an advantage, because folding and post-translational modifications may be different when using cells derived from other species. The similarity of folding and post-translational modifications is of special importance for proteins produced for medical purposes, as even minor differences may cause compatibility problems when these proteins provided to humans.

For certain applications, it is preferred that the host cells are deficient in their ability to glycosylate proteins. An example for such application is the use of proteins for X-ray diffraction purposes. This requires crystallization of the protein, which is often difficult for proteins containing for example N-linked glycans. When glycosylation deficient host cells are used for the production, the expressed proteins do not contain these glycans and can therefore be more easily crystallised. The method according to the invention is very well suitable to assess and select the most suitable expression construct in view of production of non glycosylised, or less glycosylised proteins or fragments thereof. Such host cells are e.g. known from Reeves et al., PNAS (2002) Vol. 99, No. 21, pp. 13419-13424.

More preferably, the host cells in the method are adapted to serum free medium and/or are cultured in serum free medium. ‘Serum free’ means a serum content in the culture medium of 0.4 v/v % or less, preferably 0.3 v/v % or less, preferably 0.2 v/v % or less. In serum free medium, the isolation of proteins is more convenient, as there is less contamination with serum proteins from the medium. Such cells can be obtained by step-wise limitation of the serum content. As the cells are grown in e.g. 10 v/v % FCS (foetal calf serum), the cells can be passaged into medium containing less serum, and cultured to a desired cell density. Again, the cells can be passaged into culture medium containing again less serum, etc.

Preferably, the host cells as used in the method of the invention are suspension growing cells. Suspension growing cells are easier to handle, require less working space and may give a higher protein yield than their adherently growing counterparts. Suitable cells are e.g. the HEK293 GnTI⁻ cell line as described by Reeves, supra.

Preferably, the host cells are embryonic cells, in particular human embryonic cells, more preferably human embryonic kidney cells, even more preferably HEK293 cells or cells derived thereof, such as the above described HEK293 GnTI⁻ cells (Reeves, supra). The term ‘derived thereof’ is meant to include all cells that have been developed, starting from HEK293 cell, or cells, developed there from.

Preferably, the common vector sequence comprises an origin of replication being OriP (Durocher et al., supra), and the host cells express EBNA1 (Epstein-Barr virus Nuclear Antigen 1). As discussed above, it has been shown by Durocher (supra) that cells, expressing EBNA1, are capable to produce increased amount of particular proteins by transient expression, when the genes of the said proteins are encoded on a plasmid under the control of the OriP origin of replication.

It can be attractive to provide the capacity to produce EBNA1 to cells by incorporation of the gene encoding EBNA1 on the common vector sequence used to prepare the DNA constructs for use in the method of the invention. EBNA1 may then be expressed upon transfection of the host cells with the DNA construct. To this end, the EBNA1 is encoded by the common vector sequence.

However, it is more advantageous to use host cells that have the EBNA1 encoding gene stably integrated in the genome. By this, EBNA1 can already be produced by the host cells, and is present, at the moment of transfection is performed. In case the EBNA1 encoding gene is provided on the DNA construct, it has to be expressed before it can exert its positive effect on transient gene expression. This will be demonstrated in the examples below. An example of such cells is the cell line HEK293-EBNA1 (293E) as described in WO2006/096989 (ATCC#CRL-10852).

Therefore, it is advantageous to use HEK293E cells as host cell in the method of the invention. These cells have been stably transfected with the Epstein Barr Nuclear Antigen 1 (EBNA1). The advantage of HEK293E over HEK293 is that plasmids containing the Epstein Barr virus origin of replication, OriP, are maintained episomal, rendering these cells very suitable for protein production by transient expression, making it possible for the method to be performed in a high-throughput fashion, as many different constructs can be tested in host cells in parallel. It is believed that EBNA might function as a transcription and translation enhancer that would result in higher transient expression levels compared to HEK293. The advantage of HEK293E over HEK293 is demonstrated in the examples below.

However, an SV40 ori on the plasmid, and a host cell expressing large T antigen, can also be suitable for production of proteins by transient expression.

The method according to the invention is preferably performed with novel host cells, specifically designed for use in the method according to the present invention. The cells are derived from HEK293 cells, are deficient for N-acetylglucosaminyltransferase I, and have the gene coding for EBNA1 stably integrated in their genome. In particular, the cells are HEK293GnTI⁻ES16-A cells, as deposited on Mar. 5, 2008, at the DSMZ-Deutsche Sammlung von Mikro-organismen und Zellkulturen GmbH with accession number DSM ACC2888. The said cells were obtained by starting from HEK293GnTI⁻ cells, such as described by Reeves, supra, wherein the EBNA1 gene was cloned.

As a result of the above deficiency, the cell line produces glycoproteins with only Man₅GlucNac₂ glycans. This makes these proteins excellent for e.g. X-ray diffraction, neutron diffraction and EXAFS purposes.

The above described HEK293GnTI⁻ES16-A cells are adherently growing. As outlined above, it is however advantageous to use cells that are capable of growing in suspension. To this end, another novel cell line was produced, starting from HEK293GnTI⁻ES16-A cells, and conferring to the said cells the capacity to grow in suspension, which were produced by detaching the HEK293GnTI⁻ES16-A cells from the surface of their culture container, culturing the cells in Ca²⁺-free medium, remove cell aggregates and continue culturing the cells in suspension. Therefore, in the method according to the invention the host cells are preferably suspension growing HEK293 GnTI⁻ ES16-S cells as deposited on Mar. 5, 2008, at the DSMZ-Deutsche Sammlung von Mikro-organismen und Zellkulturen GmbH with accession number DSM ACC2889.

In particular in view of protein production, the cells to be used in the method according to the invention are capable of growing in low serum or serum free media of less than 0.4 v/v % serum, preferably of less than 0.3 v/v % serum, and most preferably of less than 0.2 v/v % serum. To this end another cell line was produced, starting from the above-mentioned suspension growing HEK293 GnTI⁻ ES16-S cells. As outlined above, the cells were made suitable to grow in serum free media by successive passages of the cells in media of decreasing serum content. Accordingly, novel cell line HEK293 GnTI⁻ ES16-1S was produced.

Therefore, in the method according to the invention, the host cells are preferably suspension growing HEK293 GnTI⁻ ES16-1S cells, capable to grow in low serum medium containing 0.2% v/v serum, as deposited on Mar. 5, 2008, at the DSMZ-Deutsche Sammlung von Mikro-organismen and Zellkulturen GmbH with accession number DSM ACC2890.

This invention further relates in a further aspect to a method to produce the above-described HEK 293E GnTI⁻ cells, being deficient for N-acetylglucosaminyltransferasel, and have the gene coding for EBNA1 stably integrated in their genome. HEK293E cells, wherein the EBNA1 gene has been stably integrated in their genome, are known from Morlot et al., supra. However, Morlot suggests to use the HEK293E cell line, and to try to mutate this cell line, or to use kifunensine in the culture media in order to convert complex N-linked oligosaccharides of glycoproteins into simple Man₉(GlcNAc)₂ structures, rendering the produced proteins more suitable for crystallisation purposes.

However, mutants of HEK293E cells, deficient in glycosylation processes, have never been obtained, identified or described since. The present inventors have chosen a less straight-forward method to produce HEK293E GnTI⁻ cells, and surprisingly found the HEK 293E GnTI⁻ cells, in particular HEK 293E GnTI⁻ES16-A cells. Said method comprises the following steps:
i) culturing the HEK293 GnTI⁻ cells,
ii) transfecting cells obtained in step i) with EBNA-1
iii) culturing cells obtained in step ii),
iv) selection of HEK293E GnTI⁻ cells from the cells obtained in step iii).

In a first step, known HEK293 GnTI⁻ cells are used. These cells are immortalised human embryonic kidney cells that are deficient in N-acetylglucosaminyltransferase I. In a next step, the cultured cells are transfected with EBNA-1. Any transfection procedure can be used. A skilled person will be able to select a method that provides the best transfection efficiency. Preferably, the transfection method used in this method is performed using PEI as a transfection agent.

In a next step, cells are cultured and positive clones are selected for further use. Such methods are well known and described in the art and are described in the above-mentioned text books. Expression of EBNA1 is checked using methods that are well known in the art. An example of a procedure according to the method is provided in the examples below.

The invention also relates to HEK293 GnTI⁻E cells i.e. HEK293 derived cells, deficient in N-acetylglucosaminyltransferase I and having the EBNA1 gene stably integrated in the genome, in particular to the new cell line HEK 293 GnTI⁻ES16-A as deposited on Mar. 5, 2008, at the DSMZ-Deutsche Sammlung von Mikro-organismen und Zellkulturen GmbH with accession number DSM ACC2888, which is an adherently growing cell line.

This invention further relates to a method to confer to adherently growing HEK 293 GnTI⁻E cells, in particular HEK 293 GnTI⁻ES16-A cells, the capacity to grow in suspension for the use in the method to produce proteins or protein fragments, comprising steps of:
I. detaching adherent HEK293 GnTI⁻E cells,
II. culturing in Ca²⁺-free medium containing serum,
III. removing aggregates.

In a first step adherent growing cells are detached. There are different methods to detach adherent growing cells, for example by physical force, such as scraping the cells off the surface of the culture container, or by the use of enzymes or chemicals, such as trypsin. Any suitable detachment method may be used. A skilled person will be able to select a suitable method. In a next step, the detached cells are cultured in Ca²⁺-free medium containing serum. Subsequently, the aggregates are removed, and the non attached free cells are cultured further. The term ‘Ca²⁺-free medium’ means that the medium contains less than 25 μM Ca²⁺, preferably less than 10 μM, more preferable less than 5 μM Ca²⁺. Most preferably, the medium does not contain Ca²⁺. Suitable and preferred media are calcium free DMEM and GIBCO® FreeStyle™ 293 Expression Medium (hereafter also indicated by ‘freestyle medium’), both from Invitrogen.

The invention also relates to suspension growing HEK293E GnTI⁻E cells, in particular to the new cell line HEK293 GnTI⁻ ES16-S as deposited on Mar. 5, 2008, at the DSMZ-Deutsche Sammlung von Mikro-organismen und Zellkulturen GmbH with accession number DSM ACC2889.

These cells are similar to the previously mentioned HEK293E GnTI⁻, but differ in their capabilities to grow in suspension. Because they are adapted to suspension growth, these cells have all the benefits of suspension growing cells as mentioned earlier. These cells are therefore ideally suited for scalable production of proteins, in particular for crystallisation studies.

This invention further relates to a method to confer HEK293E GnTI⁻ cells the capacity to grow in serum free medium for the use in the method to produce proteins or protein fragments, comprising steps of:
I. Culturing the cells in medium comprising the required amount of serum for the cells to grow and replicate,
II. passaging the cells into medium having less serum than the medium from which the cells are passaged,
III. repeating step II. until the serum content is 0.4% v/v or less, preferably 0.3% v/v or less, most preferably 0.2% v/v or less.

In a first step, the cells are cultured in medium containing the amount of serum that is required. This method can be used for all adherent cell types. The amount of serum may vary between cells types. Also, the source of serum may be different, depending on the cell type that is used. Culturing conditions and medium and serum content are well known to the skilled person. In a next step, cells are passaged. This means that in case of adherent cells, the cells are detached first. There are different suitable methods to detach adherent growing cells, as discussed above. A skilled person will be able to select a suitable method. The cells are resuspended in new medium containing less serum than in step I. The cells are further cultured. In case of suspension growing cells, the cells are centrifuged, and the medium is changed, where after the cells are resuspended in medium containing less serum. Step III. is repeated until the serum content is 0.4% v/v or less, preferably 0.3% v/v or less, most preferably 0.2% v/v or less.

The invention also relates to both adherent and suspension growing HEK293 GnTI⁻ E cells, capable to grow in low serum medium of 0.4% v/v or less, preferably 0.3% v/v or less, most preferably 0.2% v/v or less, in particular suspension growing HEK293 GnTI⁻ ES16-1S cells as deposited on Mar. 5, 2008, at the DSMZ-Deutsche Sammlung von Mikro-organismen and Zellkulturen GmbH with accession number DSM ACC2890.

In another aspect, the invention relates to a method to transfect the suspension growing HEK293 GnTI⁻ E cells according to the invention, in particular HEK293 GnTI⁻ ES16-1S cells, in a medium containing a serum content of 0.1v/v %. preferably less than 0.06 v/v %, more preferably of about 0.04 v/v %, comprising steps of:
I. diluting the HEK293 GnTI⁻ E cells in a volume of serum free medium such that the medium upon dilution contains 0.1 v/v %, 0.06% or about 0.04% v/v serum, respectively,
II. transfect the diluted cells of step I.

It has been found that transfection at such low serum conditions are very effective when transfecting HEK293 GnTI⁻ E cells. The term ‘about 0.04 v/v %’ reflects an amount of 0.034-0.045 v/v % serum in the culture medium.

This invention further relates to a kit comprising at least a first and a second DNA-preconsruct suitable for use in the method of any of the claims 1-37, wherein the first and second DNA pre-constructs each comprise a common vector sequence, and a common cloning site, the first and second DNA preconstructs being different in sequence, location or presence of a DNA sequence element affecting the production of the protein or fragment thereof in an envisaged host cell. The preconstructs are ready for use in the present invention; only DNA, encoding the protein or fragment thereof, optionally including flanking coding sequences in case of a protein fragment, is still to be cloned into the preconstruct, in the common cloning site thereof.

The provision of a kit according to the invention enables convenient preparation of expression vectors and testing the suitability thereof. The features outlined above for the DNA constructs are also applicable for the preconstructs of the kit. The kit may comprise more than two different preconstructs that all differ from one another in the sequence, location or presence of one or more DNA sequence elements as described above.

The invention will now be further exemplified by referring to the figures and examples, wherein:

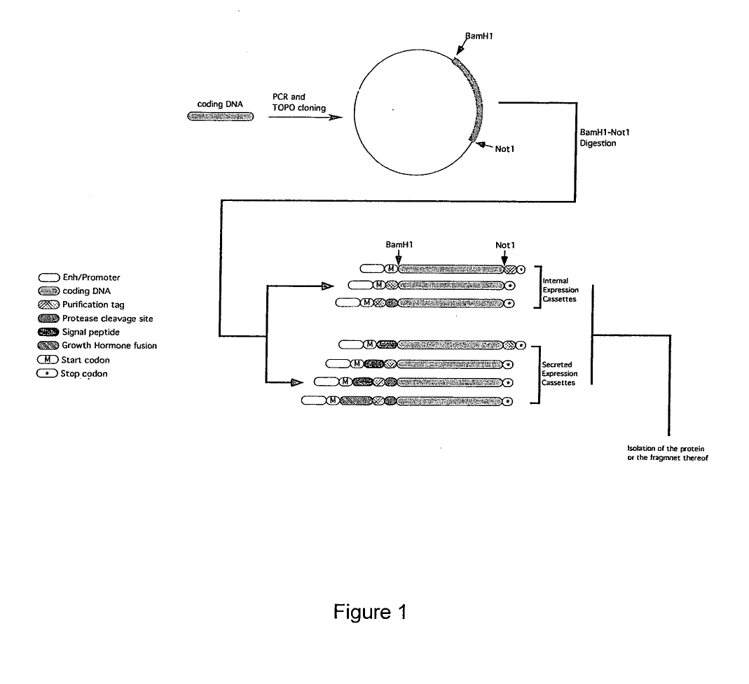

FIG. 1 shows the concept of the preparation of 7 different DNA constructs.

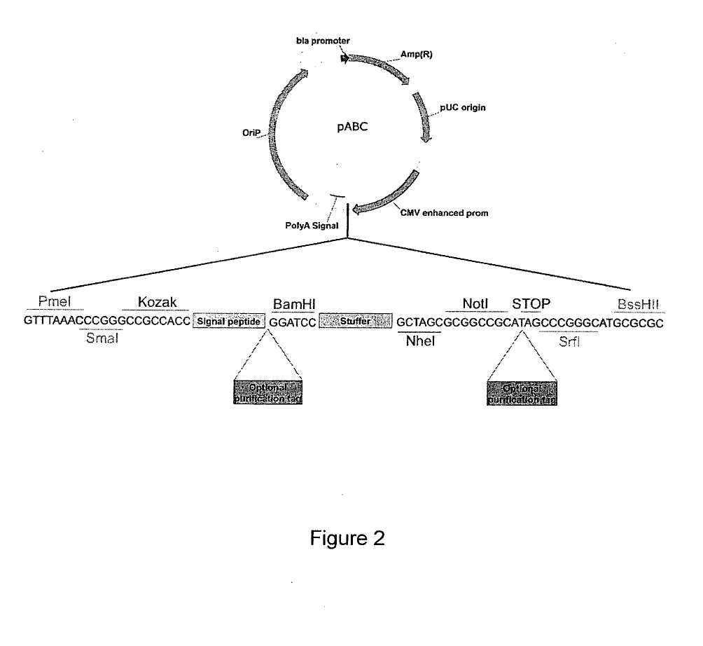

FIG. 2 is a schematic drawing of a multiple cloning site that can be used in the constructs of the present invention.

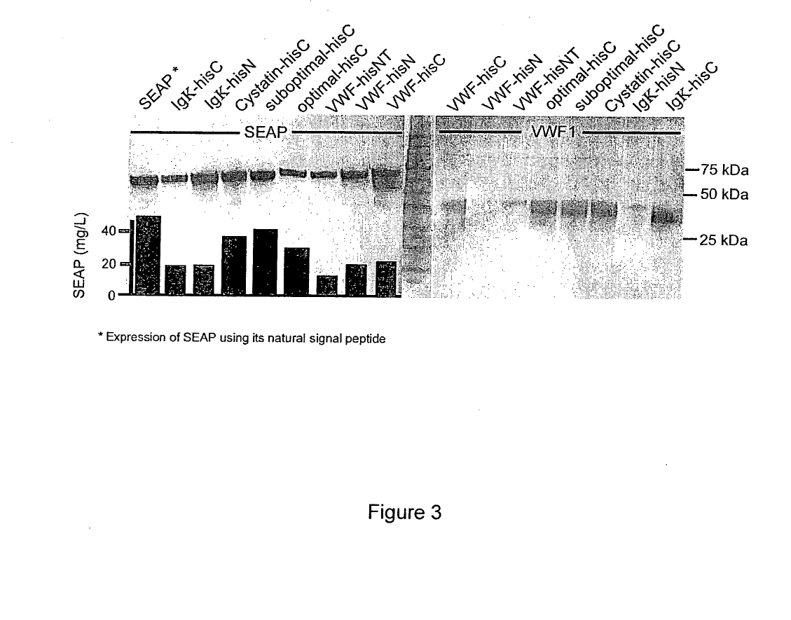

FIG. 3 shows a western blot showing the production of a model protein (placental secreted alkaline phosphatise, SEAP) and a protein fragment (Von Willebrand Factor Domain A1, VWF-A1), as expressed from different DNA constructs. The histogram shows specific SEAP activity.

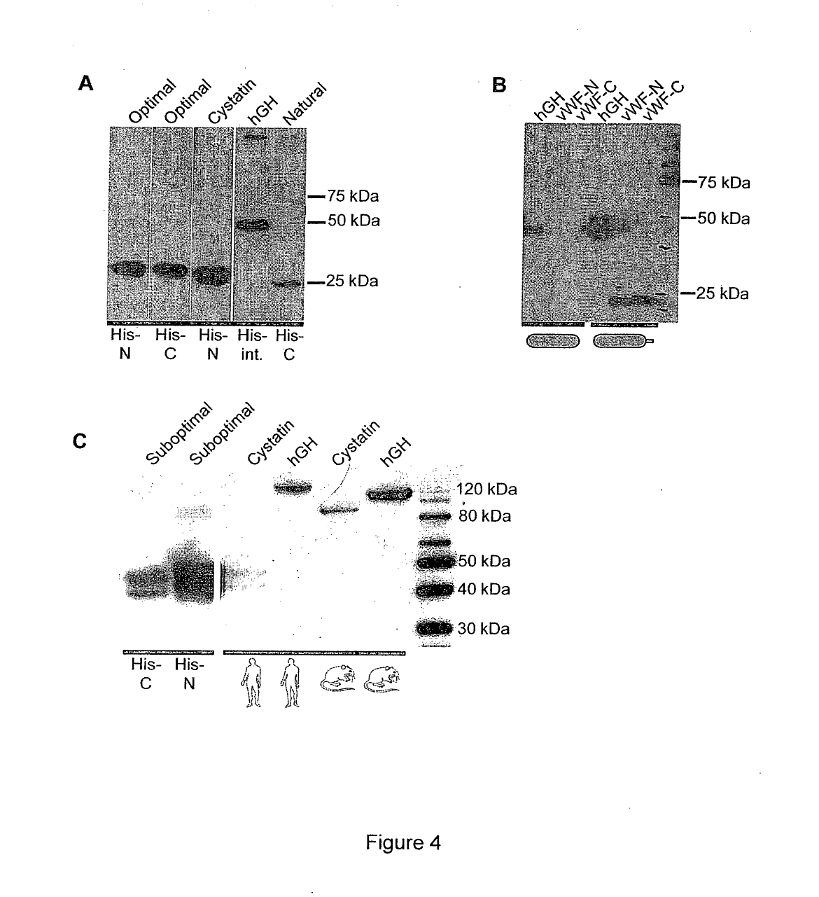

FIG. 4 shows the production of different proteins/fragments, as expressed from different DNA constructs, wherein the host cells were cultured in serum free medium.

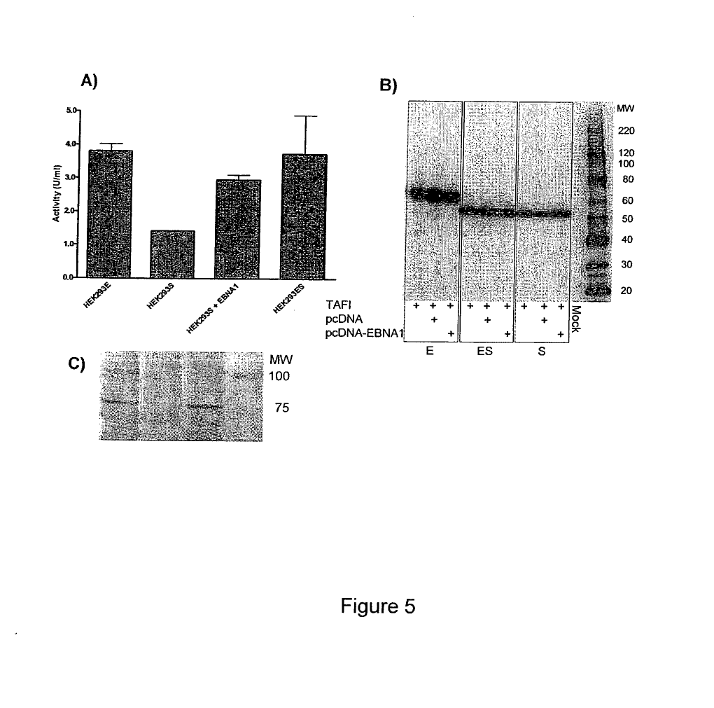

FIG. 5 shows in panel A) a histogram showing the transient expression of model protein SEAP in different HEK293 cells, Panel B) shows a western blot analysis with model protein TAFI, and panel C) shows a western blot analysis detecting the presence of EBNA-1 in the host cells with a specific antibody.

In FIG. 1, a cDNA, encoding the protein to be produced, is cloned into 7 different DNA preconstructs. The preconstructs consist mainly of a common plasmid (vector) sequence, and a common cloning site wherein a BamH1 and a Not1 restriction recognition site are present. These sites are flanking the cDNA once cloned into the preconstruct. Upstream of the BamH1 site, and downstream of the Not1 site, DNA sequence elements are located. In the upper 3 DNA constructs (i.e. the preconstruct wherein the cDNA is cloned), a polyhistidine tag sequence is located at different locations. In the upper DNA construct as well as in the fourth DNA construct 3′ of the cDNA, in the second and fifth DNA construct 5′ of the cDNA, and in the third, sixth and the seventh lowest construct, a protease cleavage site is present as additional DNA sequence element, 5′ from the cDNA, between said cDNA and the polyhistidine tag sequence.

A signal sequence is present in the fourth to seventh constructs, in the fourth directly 5′ adjacent to the cDNA, in the fifth 5′ of the polyhistidine tag sequence, in the sixth the signal peptide sequence is followed by a polyhistidine tag sequence, and a protease cleavage site being located between the said polyhistidine tag sequence and the cDNA. The seventh construct comprises the DNA sequence encoding the human growth hormone, resulting in a fusion protein with an internal polyhistidine tag and a protease cleavage site.

It is also possible, although not shown in this figure, that one or more of the DNA constructs, comprise a cDNA that encodes a fragment of a protein, accompanied by additional sequences encoding flanking portions of the said protein fragment. These portions can be adjacent sequences in the native protein, or e.g. a 5′ or 3′ terminus of the said protein.

The different constructs are used to transfect host cells, and the best producing transfected host cell is identified, and the construct, used to transfect the said host cells is selected as the suitable expression construct.

FIG. 2 shows a preconstruct, i.e. before the DNA encoding the envisaged protein to be produced is introduced, wherein the common cloning site is given in more detail. The common vector sequence comprises an OriP, an ampicillin resistance gene, a poly A signal and a CMV promoter. It is however very well possible to have the sequence of the said poly A signal and/or said CMV promoter on the DNA sequence element, that is, not present in all the constructs. For example, the presence of a CMV promoter can be tested against another promoter.

The common cloning site comprises multiple restriction endonuclease recognition sites, such as BamH1 and Not1. Upstream of the BamH1 site, and downstream of the Not1 site, a purification tag sequence can be present, such as a sequence, encoding a polyhistidine tag.

The effects of the presence of different signal peptides on the production of proteins is illustrated in the example “Effect of signal peptide on recombinant protein production in HEK293” and in FIG. 3.

FIG. 3 illustrates that the influence of the type of signal peptide used and the location of the histidine tag on the production of a protein or protein fragment is different. It shows the expression levels of two model proteins/fragments, secreted to produce proteins in HEK 293E cells, alkaline phosphatase (SEAP) and the Von Willebrand Factor A1 domain (VWF-A1), using different constructs containing different signal peptides and position of the his-tag. SEAP and VWF-A1 were cloned in different preconstructs containing different signal peptides and positions of the his-tag. Expression is analyzed by Western-blotting (SEAP and VWF-A1) and specific activity (SEAP, histogram). Expression of VWF-A1 is highly dependent on the signal peptide and location of the his-tag. In contrast, the signal peptide and location of the his-tag are of much less influence on the expression of SEAP.

SEAP: SEAP in combination with its natural signal peptide
IgK-hisC: immunoglobuline kappa signal sequence and a C terminal hexahistidine tag.
IgK-hisN: immunoglobuline kappa signal sequence and an N terminal hexahistidine tag.
Cystatin-hisC: Cystatin signal sequence and a C terminal hexahistidine tag.
Suboptimal-hisC: Suboptimal signal sequence (SEQ ID No 2) and a C terminal hexahistidine tag.
Optimal-hisC: Optimal signal sequence (SEQ ID No 1) and a C terminal hexahistidine tag.
VWF-hisNT: VWF signal sequence and a TEV cleavable N terminal hexahistidine tag.
VWF-hisN: VWF signal sequence and an N terminal hexahistidine tag.
VWF-hisC VWF signal sequence and a C terminal hexahistidine tag.

FIG. 4 shows a Westernblot (anti-His) showing the effects of different expression vectors on the secretion of specific target protein (domains).

Panel A The N-terminal extra-cellular domain of Gp1Bα was ligated in 5 pUPE expression vectors and transfected to HEK293E cells. The cystatin signal sequence (lane 3), the optimal signal sequence (lanes 1+2) and the growth hormone fusion protein (lane 4) greatly enhance secretion the Gp1Bα as compared to secretion from it's natural signal sequence (lane 5). Secretion however, is not dependent on the position of the His-tag (compare lanes 1 and 2)

Panel B The vWF-A2 domain was ligated into pUPE vectors containing the vWF signal sequence or the growth hormone fusion protein (lanes 1,2,3) secretion is only observed when directed by the growth hormone. C-terminal extension of the vWF-A1 domain with 7 residues however rescues secretion in all three-expression vectors (lanes 4,5,6)

Panel C Lanes 1 and 2 Paraoxanase 1 lacking its natural signal sequence was ligated in two pUPE expression vectors containing the suboptimal signal sequence and either a N-terminal or a C-terminal His-tag. Comparison of lanes 1 and 2 shows that secretion of paraoxanase1 is higher with a N-terminal His-tag.

Lanes 4-6 show that highly similar proteins may have different expression levels. Human and mouse C7 were ligated in two pUPE expression vectors containing either the Cystatin signal sequence or the growth hormone fusion protein. In spite of the fact that mouse and human C7 are more than 62% identical, human C7 is only secreted when the growth hormone fusion protein is used. While mouse C7 is also secreted using the cystatin signal sequence.

FIG. 5 illustrates that EBNA1 enhances protein production in HEK293-GnTI- cells. A) Plasmid pUPE-ssSEAP-hisC (see Materials and Methods section) was transiently transfected to HEK293 cells. SEAP activity was assayed using para-nitrophenylphosphate 5 days post transfection (the activity is the mean and SD of three independent transfection experiments). Transient co-transfection of pcDNA3.1-EBNA1 (see Materials and Methods section) and pUPE-ssSEAP-hisC doubles SEAP production in HEK293-GnTI- cells, whereas stable integration of EBNA1 triples SEAP production. B) Western blot analysis of HEK293E, HEK293S (‘S’ stands for GnTI⁻) and HEK293ES, lanes 1, 2 and 3, respectively. Each lane was loaded with 3.8*104 cells. EBNA1 was detected with a goat polyclonal against EBNA-1, Rabbit-anti-goat-HRP and chemiluminescence. The band at 75 kDa in HEK293E and HEK293ES is specific for EBNA1. C) Plasmid pUPE-Cystatin-HisNTEV-TAFI was transient transfected to HEK293 cells. The production of model protein TAFI (Thrombin-activatable fibrinolysis inhibitor), was assayed at 120 hours post transfection by Western blot. TAFI was detected with an anti-his-tag monoclonal antibody and Rabbit-anti-mouse-HRP. Transient co-transfection of TAFI and EBNA1 to HEK293S results in an increased TAFI production. TAFI production in HEK293ES cells does not require EBNA1 co-transfection. It is important to note that TAFI is a protein that was shown to be very difficult to be crystallised by traditional methods. Using HEK293GnTI-E (HEK293ES), the protein could be crystallized and the structure of TAFI could be resolved.

### Materials and Methods

### Media and Reagents

FreeStyle expression medium, DMEM, Ca²⁺ free DMEM, Optimem, FCS and G418 were purchased from Invitrogen. Primatone was from Kerry Bioscience. Tissue culture flasks, E-well and 24-well plates were from Greiner Bio-one. Tissue culture Erlenmeyer's were from Corning. Chemiluminescent SEAP activity assay and low melting point agarose were from Roche. All restriction enzymes and T4 DNA ligase were from New England Biolabs. Shrimp Alkaline Phosphatase was from Fermentas. Polymerase Pfu Ultra was from Stratagene. SYBR Safe nucleic acid stain and Plasmids pCRII-TOPO, pCR4-TOPO, pcDNA3.1/Neo(+) and pCEP4 were from Invitrogen. Plasmid pCI and Wizard SV gel and PCR clean-up system were from Promega. NuPage gels were from Invitrogen and PVDF was from Bio-Rad. Monoclonal anti-his-tag antibody was from Novagen. Rabbit-anti-mouse-peroxidase was from Sigma. Polyclonal anti-EBNA-1 and Rabbit-anti-goat-peroxidase were from Abcam. Enhanced chemiluminescence kit was from GH-Healthcare. Spin miniprep kit was from Qiagen and the Genelute maxiprep kit and paranitrophenylphosphate were from Sigma. All other chemicals were from Merck.

Construction of pcDNA3.1-EBNA-1

The Open Reading Frame of EBNA1 was amplified from plasmid pCEP4 by PCR using oligo's as described in table I. The EBNA-1 PCR fragment was ligated into pCRII-TOPO vector, generating pCRII-TOPO-EBNA1. Two positive clones were sequenced and the BamHI-EcoRI fragment of the clone that contained the correct sequence was ligated into pcDNA3.1/Neo(+) generating pcDNA3.1/Neo-EBNA1. The presence of EBNA1 in the vector was confirmed by BamHI-NotI restriction analysis.

TABLE I
Oligonucleotides

| Oligo | protein | Nucleotide sequence (5′ > 3′)¹ |
|---|---|---|
| EBNA-F | EBNA | gga tcc GAT GTC TAT TGA TCT |
| | | CTT TTA GTG TG |
| EBNA-R | EBNA | gaa ttc GCT TTT AAT ACG ATT |
| | | GAG GGC G |
| TAFI-F | TAFI | gaa gat ctT TTC AGA GTG GCC |
| | | AAG TTC |
| TAFI-R | TAFI | ata gtt tag cgg ccg cTT AAA |
| | | CAT TCC TAA TGA CAT G |
| ss-SEAP-F² | SEAP | aga tct gcc gcc acc ATG CTG |
| | | GGC CCC TGC ATG CTG CTG CTG |
| | | CTG CTG CTG CTG GGC CTG AGG |
| SEAP-F³ | SEAP | aga tct ATC ATC CCA GTT GAG |
| | | GAG GAG AAC CCG G |
| SEAP-R | SEAP | gcg gcc gcA CCC GGG TGC GCG |
| | | GCG TCG G |

¹Non annealing parts are shown in lower case
²For expression of SEAP with its native signal peptide
³For expression of SEAP with a signal peptide from the pUPE expression vector

Construction of pUPE Expression Vectors for Protein Production in HEK293EBNA1 Cells

pUPE is a consecutive combination of the following fragments: 1) the BgIII-NheI fragment of pCI containing the Cytomegalovirus immediate-early enhancer/promoter region; 2) the NotI-BsmBI fragment of pCEP4 containing the SV40 PolyA and the Epstein-barr virus Origin of replication OriP; 3) The SalI (blunt)-SalI (blunt) fragment of pcDNA3.1(+), containing Amp(R) and pUC Origin of replication; 4) a multiple cloning site as shown in FIG. 2 was cloned in the NheI (blunt) and NotI (blunt) sites between the CMV promoter/enhancer and the SV40 polyA. Signal sequences were based on a study of Barash et al. (2002) Biochem. Biophys. Res. Comm. Vol. 294, No. 4, pp. 835-842 (table II). Alternatively, the signal peptide was replaced by an ATG codon in expression vectors for internal protein production. pUPE expression plasmids were constructed without or with either N- or C-terminal-tags for recognition and/or purification purposes. A Kozak sequence was included as well (Kozak (2005) Gene, Vol. 361, pp. 13-37).

TABLE II
signal peptides

| Signal peptide | Amino acid sequence |
|---|---|
| Optimal¹ | MWWRLWWLLLLLLLLWPMVWA |
| Sub-optimal¹ | MRPWTWVLLLLLLICAPSYA |
| Cystatin S¹ | MARPLCTLLLLMATLAGALA |
| IgKappa | METDTLLLWVLLLWVPGSTGD |

¹based on a study by Barash et al., supra.

### Construction of SEAP and TAFI Expression Vectors

Plasmids pTT3-SEAP (Durocher, supra.) and pCRII-TOPO-TAFI (PF Marx et al, J. Biol. Chem., 2000, 275 (17), 12410-12415) were used as a template for a PCR reaction with the gene oligonucleotides as described in table I. The A-tailed PCR fragment was ligated in pCR4-TOPO, generating pCR4-TOPO-SEAP and pCR4-TOPO-TAFI, respectively. The presence of SEAP and TAFI in the pCR4-TOPO vectors was confirmed by restriction analysis using restriction enzymes BgIII and NotI. The sequence of positive clones was confirmed by DNA sequencing. Next, the BgIII-NotI fragment containing SEAP or TAFI was cloned in pUPE expression vectors and the presence of SEAP or TAFI was confirmed by restriction analysis.

### Generation of HEK293EBNA-GnTI⁻ Cell Lines

Adherent HEK293-GnTI⁻ cells were expanded to 80% confluence in 6 well plates containing 3 ml 90% DMEM+10% FCS. Cells were transfected with plasmid pcDNA3.1/Neo(+)-EBNA-1 that was complexed with polyethyleneimine ten minutes before transfection. Twenty-four hours post transfection the cells were trypsinized and suspended in 90% DMEM 10% FCS medium containing 400 μg/ml G418. Individual clones were scraped from the Petri dishes after 2-3 weeks and subsequently expanded in 24 well-plates and 6 well plates. Since we aimed at generating a highly transfectable protein production cell line—which is not necessarily linked to the highest EBNA1 expression levels—we decided in the next selection step not to first screen the obtained clones for the presence of the EBNA1 protein. Each clone was however seeded in duplicate wells of a six well plate and the wells were separately transfected with expression vectors pUPE-SEAP+oriP or pUPE-SEAP-deloriP. SEAP expression levels were monitored at regular intervals using the luminescent SEAP activity assay. Clones were selected that showed high SEAP expression levels when transfected with pUPE-SEAP+oriP and a high difference ratio, when comparing SEAP expression levels from both expression vectors. This ratio is deemed to be indicative for the recombinant protein production enhancing effect of the oriP EBNA1 combination

From the adherent cell clones the best performing one was selected, amplified, aliquoted, stored in liquid nitrogen and deposited as HEK 293 GnTI⁻ES16-A on Mar. 5, 2008, at the DSMZ-Deutsche Sammlung von Mikro-organismen und Zellkulturen GmbH with accession number DSM ACC2888.

From these cells, suspension growing cells were selected by culturing the cells in 45% Ca-free DMEM, 45% Freestyle, 10% FCS, 50 μg/ml and stepwise dilution to 70% Freestyle, 27% Ca-free DMEM, 3% FCS 50 μg/ml G418, with occasional trypsinisation until cell aggregates disappeared from the culture medium. These cells were deposited as HEK293 GnTI⁻ ES16-S on Mar. 5, 2008, at the DSMZ-Deutsche Sammlung von Mikro-organismen und Zellkulturen GmbH with accession number DSM ACC2889.

A serum free growing cell line was subsequently generated by first seeding the cells at 0.5 cell/well in 24 well plates and adapting the surviving clone by stepwise dilution to 99.8 Freestyle 0.2% FCS 50 μg/ml G418. These cells are deposited as HEK293 GnTI⁻ ES16-1S, on Mar. 5, 2008, at the DSMZ-Deutsche Sammlung von Mikro-organismen und Zellkulturen GmbH with accession number DSM ACC2890.

### HEK293 Culture Conditions

HEK293 suspension cells were routinely cultured in 1 L polycarbonate tissue culture Erlenmeyer's in FreeStyle medium, containing 0.2% serum. For HEK293ES cells, 50 μg/ml G418 was added as well. The Erlenmeyer's were placed in a humidified shaking incubator (Thermo Scientific) at 5% CO₂ and 37° C. Cell density was maintained between 0.2 and 1.5*10⁶ cells/ml. Cell density, viability and aggregation number were determined with the Casy counter (Scharfe Instruments).

### Small Scale HEK293 Transient Transfection Conditions

High quality miniprep plasmid DNA of an expression plasmid was isolated from Top10 E. coli cells from a 5 ml LB culture using the QIAprep spin miniprep kit (Qiagen). Typical yields were 20 μg for each isolation.

Twenty-four hours before transfection HEK293 cells were diluted with FreeStyle medium without additives to 0.25*10⁶ cells/ml in a 500 ml polycarbonate Erlenmeyer. The next day cells were seeded in 6-wells plates, 4.0 ml/well, and were transfected with 2.0 μg plasmid DNA that was complexed with 4.0 μg polyethyleneimine in 100 μl Optimem ten minutes before transfection. Four hours post transfection 0.9% Primatone was added. Protein production was monitored at regular intervals until 144 hours post transfection.

### Large Scale HEK293 Transient Transfection Conditions

High quality maxiprep plasmid DNA of an expression plasmid was isolated Top10 E. coli cells from a 200 ml LB culture using the GenElute HP plasmid maxiprep kit from Sigma. Typical yields were 1.5 mg for each isolation.

Twenty-four hours before transfection HEK293 cells were diluted with FreeStyle medium to 0.25*10⁶ cells/ml in a final volume of 1 L in a 3 L polycarbonate Fernbach Culture Flask. The next day the cells were transfected with 0.50 mg plasmid DNA that was complexed with 1.0 mg polyethyleneimine in 25 ml Optimem ten minutes before transfection. Four hours post transfection 0.9% Primatone was added. Expression medium was harvested 144 hour post transfection.

### SEAP Activity Assays

The activity of SEAP was determined by either the chemilumenescent SEAP reporter gene assay method (Roche) according to the manufacturers' recommendations or by the pNPP assay. In this assay, 3.2 mM para-nitrophenylphosphate was used as a substrate in a buffer containing 9 mM MgCl₂, 25 mM glycine pH 9.6. Samples of the conditioned culture media (containing recombinant SEAP) were incubated with 950 μl assay buffer and the increase in absorbance at 405 nm was recorded for 30 seconds. SEAP activity was expressed as dA/min.

### Purification of TAFI From Conditioned Medium

Recombinant TAFI was purified from a 4 L culture of HEK293ES cells that has been transfected with TAFI that was cloned into an appropriate pUPE expression vector. One hundred forty-four hours post transfection conditioned medium was collected by centrifugation (1000 g, 30 minutes, 4° C.). The conditioned medium was concentrated 10 fold using a Quixstand hollow fiber system (GE-healthcare) and a 10 kDa cartridge followed by diafiltration against 4 L 25 mM Tris 500 mM NaCl pH 8.2. Debris was removed by filtration over a glass filter (Satorius) and 5 mM imidazol was added. Fifty ml aliquots were stored at −20° C. until use. TAFI was purified from 2 aliquots by batch binding to 1.0 ml nickel sepharose FF (GE-healthcare) for 2 to 3 h at RT. Bound TAFI was eluted with 125 mM imidazol. Immediately after elution TAFI was further purified by immuno-affinity using monoclonal 9H10 that coupled to CNBr-activated Sepharose column. The column was equilibrated with 50 mM Tris, 150 mM NaCl, pH 7.4. Unbound and non-specifically bound proteins were washed away with 50 mM tris 500 mM NaCl. Bound TAFI was eluted with 0.1 M glycine, pH 4.0. Elution fractions were collected in 1/200 (v/v) 1 M Tris, pH 9 and pooled. TAFI appeared as a single band on a silver stained gel.

### Protein Electrophoresis and Western Blotting

Protein samples were made in NuPage reducing sample buffer. NuPage gels (4-12%) were used. Proteins were stained with coomassie or were transferred to PVDF. EBNA-1 was detected with polyclonal α-EBNA-1 and rabbit-anti-goat-HRP. His-tagged proteins were detected with α-his-tag antibody and rabbit-anti-mouse-HRP.

### Results

Effect of EBNA-1 on protein production in HEK293-GnTI⁻ cells

One ampoule of adherent HEK293-GnTI⁻ cells was seeded in a 75 cm² tissue culture flask in DMEM medium containing 5% FCS. At 90% confluence cells were detached by trypsinization and seeded in 20 ml Ca²⁺-free DMEM containing 5% FCS in a 125 ml Erlenmeyer. Initially HEK293-GnTI⁻ cells grew slowly in suspension and formed aggregates. Aggregates were isolated from the suspension culture, trypsinized and single cells were added back into the suspension culture. In 10-12 weeks the cells adapted to suspension conditions, did not form aggregates and had a generation time of 24-30 hours. In the next 12 weeks FreeStyle medium was gradually titrated into the medium (0.9 v/v). HEK293-GnTI⁻ cells were adapted to low-serum conditions by gradual reduction of FCS to 0.2% v/v. Finally Ca²⁺-free DMEM was completely omitted from the medium. Generation time of HEK293-GnTI⁻ suspension cells in FreeStyle medium containing 0.2% FCS is 20-24 hours. Aliquots were stored in liquid nitrogen for future use.

To study the effect of EBNA-1 on protein production in HEK293-GnTI⁻ cells, the cells were transfected with pUPE-ssSEAP-hisC or co-transfected with pUPE-ssSEAP-hisC and pcDNA3.1-EBNA-1 in 4 ml cultures in a 6-wells plate. At regular intervals 100 μl samples were taken. Cells were removed by centrifugation (1 minute, 1000 g) and supernatants were stored at 4° C. SEAP production was monitored by the pNPP activity assay (FIG. 5a). SEAP production in HEK293-GnTI⁻ was 2-fold higher in the presence of EBNA-1 as has been shown for HEK293 cells before (Durocher, supra).

To standardize protein production in HEK293-GnTI⁻ cells in the presence of EBNA-1, adherent HEK293-GnTI⁻ cells were stably transfected with pcDNA3.1/neo(+)-EBNA-1. Transfectants were selected by growth on G418 containing medium and well growing, EBNA1 expressing cells were selected by comparing SEAP expression levels after transfection of selected clones with plasmids. pUPE-SEAP+oriP or pUPE-SEAPdel oriP (see materials & methods section). The clone (HEK 293 GnTI⁻ES16-A) selected by this method was in subsequent steps adapted to suspension growth (giving HEK293 GnTI⁻ ES16-S) and later also to serum free suspension growth, giving cell line HEK293 GnTI⁻ ES16-1S. The presence of EBNA-1 in HEK293ES was also confirmed by western blotting using a polyclonal antibody directed against EBNA-1 (FIG. 5b).

To demonstrate that HEK293ES had the combined phenotypes of HEK293E and HEK293-GnTI⁻, the three cell lines were transiently transfected with SEAP and TAFI (FIGS. 5a and 5c, respectively). Indeed, SEAP production levels of HEK293ES were three-fold higher compared to HEK293-GnTI⁻ and are similar to the SEAP production levels of HEK293E.

1-51. (canceled)

52. Method for selecting a suitable expression construct from a plurality of expression constructs for optimizing the production of a protein or a fragment thereof in a host cell, the fragment not being a Slit2 LRR domain, comprising the following steps:
a) providing a first and a second DNA construct, each comprising
a common vector sequence,
a common cloning site,
a DNA encoding the protein or fragment thereof,
the constructs being different in sequence, location or presence of a DNA sequence element affecting the production of the protein or fragment thereof by the envisaged host cell;
b) providing host cells, and transfecting a first portion of the host cells with the first construct obtained in step a), resulting in first transfected host cells, and transfecting a second portion of the host cells with the second construct obtained in step a), resulting in second transfected host cells;
c) culturing the transfected host cells of step b) under conditions allowing the production of the protein or fragment thereof by the transfected host cells;
d) determining the amount and/or quality of the protein or fragment thereof, produced by the first and second transfected host cells;
e) selecting the host cells producing the highest amount or quality of the protein or protein fragment as determined in step d); and
f) selecting the DNA construct used for transfection of the host cells as selected in step e) as the suitable expression construct.

53. Method according to claim 52, wherein in step a) n different expression constructs are provided, in step b) n portions of the host cells are provided, which are transfected with the n expression constructs, resulting in n different transfected host cell portions, and in step d) the amount and/or quality of the protein or fragment thereof, produced by the n different transfected host cell portions is determined, n being 3 or more.

54. Method for the production of a protein or fragment thereof, comprising the following steps:
I. transfecting host cells with a construct, selected according to claim 52;
II. culturing the transfected host cells under conditions allowing the production of the protein or fragment thereof in the transfected host cells; and
III. harvesting the produced protein or fragment thereof from the transfected host cells of step II.

55. Method according to claim 54, wherein the protein or protein fragment is produced by the host cells by transient expression of the DNA encoding the protein or fragment thereof.

56. Method according to claim 52, wherein the DNA sequence element is located adjacent to the DNA encoding the protein or fragment thereof, and encodes an amino acid sequence element so that, when the protein or fragment thereof is produced by the host cells, the said amino acid sequence element is linked to the protein or fragment thereof.

57. Method according to claim 56, wherein the amino acid sequence element comprises a signal peptide.

58. Method according to claim 56, wherein the amino acid sequence element comprises a detection/purification tag, preferably chosen from the group consisting of histidine tag, affinity tag, immuno affinity tag, fluorescent label.

59. Method according to claim 56, wherein the production of protein or fragment thereof is limited to a fragment of the said protein, and wherein the amino acid sequence element corresponds to a portion of the same protein, so that the said protein fragment, when produced by the host cells, is linked to the said portion.

60. Method according to claim 52, wherein the host cells are human cells.

61. Method according to claim 52, wherein the host cells are deficient in glycosylation.

62. Method according to claim 61, wherein the host cells are embryonic cells.

63. Method according to claim 52, wherein the vector sequence comprises an origin of replication being OriP, and wherein the host cells express EBNA1.

64. Method according to claim 63, wherein the EBNA1 is encoded by the vector sequence.

65. Method according to claim 52, wherein the host cells are selected from the group consisting of:
i. HEK293E cells;
ii. cells being derived from HEK293 cells, deficient for N-acetylglucosaminyltransferase I, and having the gene coding for EBNA1 stably integrated in their genome (HEK293GnTI⁻E cells), in particular being adherent growing HEK 293 GnTI⁻ES16-A cells as deposited on Mar. 5, 2008, at the DSMZ-Deutsche Sammlung von Mikro-organismen und Zellkulturen GmbH with accession number DSM ACC2888;
iii. cells being suspension growing HEK293 GnTI⁻E cells, in particular HEK293 GnTI⁻ ES16-S cells as deposited on Mar. 5, 2008, at the DSMZ-Deutsche Sammlung von Mikro-organismen und Zellkulturen GmbH with accession number DSM ACC2889; and
iv. cells being suspension growing HEK293 GnTI⁻E cells, capable to grow in low serum medium containing 0.4 v/v % or less, preferably 0.3 v/v % or less, most preferably 0.2 v/v % or less serum, in particular HEK293 GnTI⁻ES16-1S cells as deposited on Mar. 5, 2008, at the DSMZ-Deutsche Sammlung von Mikro-organismen and Zellkulturen GmbH with accession number DSM ACC2890.

66. Method for the production of a protein or fragment thereof, comprising the following steps:
a. providing a first and a second DNA construct, each comprising
a common vector sequence,
a common cloning site,
a DNA encoding the protein or fragment thereof,
the constructs being different in sequence, location or presence of a DNA sequence element affecting the production of the protein or fragment thereof by the envisaged host cell;
b. providing host cells, and transfecting a first portion of the host cells with the first construct obtained in step a), resulting in first transfected host cells, and transfecting a second portion of the host cells with the second construct obtained in step a), resulting in second transfected host cells;
c. culturing the transfected host cells of step b) under conditions allowing the production of the protein or fragment thereof by the transfected host cells;
d. determining the amount and/or quality of the protein or fragment thereof, produced by the first and second transfected host cells;
e. selecting the host cells producing the highest amount or quality of the protein or protein fragment as determined in step d);
f. culturing the selected host cells of step e) of under conditions allowing the production of the protein or protein fragment in the host cells; and
g. harvesting the protein or protein fragment produced by the selected host cells.

67. Method according to claim 66, wherein the protein or protein fragment is produced by the host cells by transient expression of the DNA encoding the protein or fragment thereof.

68. Kit, comprising at least a first and a second DNA-preconsruct suitable for use in the method of claim 52, wherein the first and second DNA-preconstructs each comprise a common vector sequence, and a common cloning site, the first and second DNA-preconstructs being different in sequence, location or presence of a DNA sequence element affecting the production of the protein or fragment thereof in an envisaged host cell.

69. Kit according to claim 68, wherein the DNA sequence element of at least one of the preconstructs comprises a signal peptide, preferably selected from the group comprising Cystatin S, IgK, VWF, BiP and an artificial signal peptide having a protein sequence according to SEQ ID NO:1 or SEQ ID NO: 2.

70. Cells selected from the group consisting of:
i. adherently growing HEK 293 GnTI⁻E cells, in particular HEK 293 GnTI⁻ES16-A cells as deposited on Mar. 5, 2008, at the DSMZ-Deutsche Sammlung von Mikro-organismen und Zellkulturen GmbH with accession number DSM ACC2888;
ii. suspension growing HEK293 GnTI⁻ E cells, in particular HEK293 GnT⁻ ES 16-S cells as deposited on Mar. 5, 2008, at the DSMZ-Deutsche Sammlung von Mikro-organismen und Zellkulturen GmbH with accession number DSM ACC2889; and
iii. suspension growing HEK293 GnT⁻ E cells, capable to grow in low serum medium of 0.4% or less, preferably 0.3 v/v % or less, most preferably 0.2% v/v % or less, in particular HEK293 GnTI⁻ ES16-1S cells as deposited on Mar. 5, 2008, at the DSMZ-Deutsche Sammlung von Mikro-organismen und Zellkulturen GmbH with accession number DSM ACC2890.

71. Method to transfect the suspension growing HEK293 GnTI⁻ E cells according to claim 70 in a medium containing a serum content of 0.1v/v %, preferably less than 0.06 v/v %, more preferably of about 0.04 v/v %, comprising steps of:
I. diluting the HEK293 GnTI⁻ E cells in a volume of serum free medium such that the medium upon dilution contains 0.1 v/v %, 0.06% or about 0.04% v/v serum, respectively; and
II. transfect the diluted cells of step I.
